(19) United States
(12) Patent Application Publication
(10) Pub. No.: US 2026/0216248 A1
(43) Pub. Date: Jul. 30, 2026

(54) CELL-MICROSPHERE CO-ASSEMBLY, AND PREPARATION METHOD THEREFOR AND USE THEREOF

(71) Applicant: WEST CHINA HOSPITAL, SICHUAN UNIVERSITY, Chengdu, Sichuan (CN)

(72) Inventors: Mao CHEN, Chengdu, Sichuan (CN); Ying HAO, Chengdu, Sichuan (CN); Yuwen CHEN, Chengdu, Sichuan (CN)

(21) Appl. No.: 19/151,899

(22) PCT Filed: Jun. 29, 2023
(86) PCT No.: PCT/CN2023/103669

§ 371 Date: Jul. 29, 2025

(30) Foreign Application Priority Data
Jun. 8, 2023 (CN) 202310677082.2

(51) Int. Cl. A61K 35/28; A61K 9/00; A61K 9/16; A61K 35/32; A61K 35/34; A61P 9/10

(52) U.S. Cl. CPC A61K 35/28; A61K 9/0019; A61K 9/1617; A61K 9/1664; A61K 35/32; A61K 35/34; A61P 9/10

(57) ABSTRACT

A cell-microsphere co-assembly is formed by means of co-assembling hydrogel microspheres and cells. The ratio of the hydrogel microspheres to the cells is 0.0001-10 μL:10¹⁻⁷ cells in the microsphere-cell co-assembly. The microsphere-cell co-assembly enhances the paracrine effect of cells and can be injected into a damaged part to facilitate the repair of damaged cells.

### FIELD OF THE INVENTION

The present invention specifically relates to a cell-microsphere co-assembly, a preparation method therefor, and uses thereof.

### BACKGROUND OF THE INVENTION

Damage to tissues and organs such as the cardiac muscle, skin, muscles, chondrocytes, blood vessels, tendons, and nerves seriously endangers the physical and mental health of patients. With the acceleration of China's aging society, the incidence rate and mortality rate are increasing year by year. Since most human tissues and organs do not have regenerative capabilities, it is crucial to promote the repair of damaged tissues and organs.

In recent years, stem cell therapy, primarily utilizing mesenchymal stem cells (MSCs), has demonstrated promising therapeutic effects in treating tissue and organ injuries. MSCs possess advantages such as low immunogenicity and a wide range of sources, enabling them to promote the repair of damaged tissues and organs with aid of sustained paracrine effects. However, due to the bad microenvironment in the injury area, the local retention and survival rates of MSCs are low, limiting their therapeutic efficacy.

Hydrogel microspheres refer to spheres or quasi-spheres with a particle size in the micrometer range, which can be used to deliver bioactive substances such as medicaments, genes, proteins, and factors. After local injection, the hydrogel microspheres undergo slow degradation, and the loaded bioactive substances will diffuse and be slowly released locally according to the degradation of the microspheres, with the advantages of long-acting, safety, and targeting. Thereby, they have broad application potential in clinical treatment. In addition, compared with the traditional hydrogel, the microsphere system has a larger specific surface area and can better interact with the surrounding microenvironment. Jian Zhou et al., Properties of gelatin methacrylate/decellularized meniscus extracellular matrix composite hydrogel with different crosslinking densities [J], Chinses Journal of Tissue Engineering Research, 2493-2499, have disclosed a hydrogel microsphere loaded with cells, but it is only loaded with cells by photo-crosslinking, resulting in a relatively fixed size of the composite hydrogel. Moreover, due to the limited cell viability, it could not interact well with the external environment, affecting its efficacy. It is necessary to develop a gel composite with controllable size, good interaction with the external environment, paracrine function, and the ability to continuously maintain the secretion of relevant repair factors.

### CONTENT OF THE INVENTION

To address the above issues, the present invention provides a cell-microsphere co-assembly, which is formed by the co-assembly of hydrogel microspheres and cells; the ratio of the hydrogel microspheres to cells is 0.0001-10 μL:10¹⁻⁷ cells;
the hydrogel microspheres are microspheres prepared by mixing any one or more of synthetic materials, natural materials, or derivative materials with photoinitiators;
the synthetic materials comprise polyethylene glycol, polyvinyl alcohol, povidone, polylactic acid, polycaprolactone, polyurethane, or polyglycerol sebacate;
the natural materials comprise gelatin, sodium alginate, sodium hyaluronate, chitosan, fibrin, collagen, or decellularized extracellular matrix (dECM);
the derivative materials comprise poly(ethylene glycol) diacrylate (PEGDA), methacryloylated poly(vinyl alcohol), methacryloylated polycaprolactone, methacryloylated polylactic acid, polyurethane methacrylate, gelatin methacryloyl (GelMA), sodium methacryloylated hyaluronate, sodium methacryloylated alginate, chitosan methacryloyl (CSMA), methacryloylated fibrin, methacryloylated collagen, or methacryloylated decellularized extracellular matrix.

Further, the hydrogel microspheres are microspheres prepared by mixing any one or more of synthetic materials, natural materials, or derivative materials with photoinitiators by means of microfluidic method, solvent evaporation method, phase separation method, spray drying method, electrostatic spraying method, templating method, or suspension polymerization method.

More further, the hydrogel microspheres are microspheres prepared by mixing methacryloylated decellularized extracellular matrix with photoinitiators and then passing through microfluidic chips.

More further, the methacryloylated decellularized extracellular matrix is prepared by reacting the decellularized extracellular matrix with methacrylic anhydride under alkaline conditions, dialyzing the reaction product, and lyophilizing it; the photoinitiators comprise lithium phenyl-2,4,6-trimethylbenzoylphosphinate.

More further, the decellularized extracellular matrix is derived from porcine myocardial tissue.

Further, the ratio of the hydrogel microspheres to cells is 10 μL:10⁶ cells.

More further, the cells comprise any one or more of mesenchymal stem cells, pluripotent stem cells, cardiomyocytes, vascular endothelial cells, adipose-derived stem cells (ADSCs), chondrocytes, and tendon cells, and mesenchymal stem cells are preferable.

Further, the particle size of the cell-microsphere co-assembly is 1-2000 μm; the particle size of the hydrogel microspheres is 1-1000 μm.

The present invention also provides a method for preparing the cell-microsphere co-assembly mentioned above, which comprises the following steps:
1) Any one or more of synthetic materials, natural materials, or derivative materials are allowed to mix with a photoinitiator aqueous solution, and then passed through a microfluidic chip to obtain hydrogel microspheres;
2) The hydrogel microspheres obtained in step 1) are mixed with cells according to the pre-determined ratio for co-assembly, to provide the cell-microsphere co-assembly.

Further, in step 1), the decellularized extracellular matrix is dissolved in water, to which is added methacrylic anhydride, and the reaction is carried out under alkaline conditions. The reaction product is dialyzed and lyophilized to obtain the methacrylated decellularized extracellular matrix, which is then mixed with the photoinitiator aqueous solution and passed through microfluidic chip to obtain hydrogel microspheres;
more further, in step 1), the mass-to-volume ratio of the decellularized extracellular matrix, water, and methacrylic anhydride is 1 g:100 mL:1 mL; the mass-to-volume ratio of the methacrylated decellularized extracellular matrix, photoinitiator, and water is 10 g:100-600 mg:100 mL; the photoinitiators comprise lithium phenyl-2,4,6-trimethylbenzoylphosphinate.

Further, in step 1), the pH value of the alkaline solution is 8-12; the reaction temperature is 0 ° C., and the reaction time is 1-48 h; dialysis is performed in water at 0-50° C., using 3500 dialysis bag; the methacrylated decellularized extracellular matrix is mixed with an aqueous solution of photoinitiator and passed through a microfluidic chip as the disperse phase and continuous phase; the flow rate ratio of the disperse phase to the continuous phase is 1:1-1:60, and the flow rate ratio of 1:20 is preferable; the continuous phase is a liquid paraffin solution containing 1-30% Span 80.

Further, in step 2), the co-assembly involves a duration of 18-36 h at a temperature ranging from 35° C. to 40° C., with a carbon dioxide concentration of 3-8%. The culture medium is low-glucose DMEM medium containing 10% FBS and 1% penicillin-streptomycin solution.

More further, the penicillin-streptomycin solution consists of 10000 units/mL of penicillin and 10 mg/mL of streptomycin.

The present invention also provides the use of cell-microsphere co-assembly mentioned above in the manufacture of a medicament for tissue repair.

Further, the tissue comprises cardiac muscle, skin, muscle, chondrocytes, blood vessels, tendons, and/or nerves.

Further, the medicament is that for the treatment of myocardial infarction.

More further, the medicament has the effect of improving cardiac function, reducing the infarct size, and/or decreasing fibrosis in the infarct area.

Further, the medicament is a local injection; the local injection has the effects of promoting neovascularization, inhibiting cell apoptosis, and accelerating tissue repair by locally injecting it into the damaged site, utilizing the paracrine function of cells.

Further, the local injection comprises intramyocardial injection, pericardial cavity injection, coronary artery injection by catheter, intradermal injection, subcutaneous injection, intramuscular injection, joint cavity injection, vascular cavity injection, tendon sheath injection, intrathecal injection, and/or spinal nerve injection.

The microsphere-cell co-assembly of the present invention, which is made by co-assembling microspheres prepared from specific components with mesenchymal stem cells, can provide an excellent microenvironment for cells and enhance their paracrine effects. After being injected in situ into the damaged site, the co-assembly can improve the therapeutic efficiency of stem cells.

Experimental results have demonstrated that the microsphere system of the present invention can promote cell proliferation and migration, and facilitate angiogenesis and inhibit apoptosis of damaged cells via paracrine mechanisms. This provides a new and more effective option for promoting the repair of damaged tissues and for clinical treatment of tissue and organ injuries.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Microscopic image of ECM microspheres (scale bar: 50 μm);

FIG. 2. (A) Transmission electron microscopy (TEM) photos (scale bar: 5 μm, 500 nm) and (B) microscopic image (scale bar: 100 μm) of ECM-MSC;

FIG. 3. The survival status of MSC cells in ECM-MSC;

FIG. 4. The relationship between the amounts of cells and microspheres and the size of the cell-microsphere co-assembly;

FIG. 5. Angiogenesis experiment on ECM-MSC (scale bar: 100 μm);

FIG. 6. Cell migration experiment on ECM-MSC (MSC: mesenchymal stem cells; EMB: control microspheres; EMA: ECM-MSC co-assembly);

FIG. 7. After treatment with ECM-MSC, (A) Echocardiogram, sham: control group, MI: myocardial infarction group, ECM-MSC: cell-microsphere co-assembly group, (B) ejection fraction, and (C) shortening fraction;

FIG. 8. TCC staining image, sham: control group, MI: myocardial infarction group, ECM-MSC: cell-microsphere co-assembly group;

FIG. 9. (A) H&E staining images and (B) Masson staining images.

### EXAMPLES

### Example 1 Preparation of Cell-Microsphere Co-Assemblies According to the Present Invention

1) 0.05 g of decellularized extracellular matrix derived from porcine myocardial tissue was dissolved in 5 mL of water, to which was added 0.05 mL of methacrylic anhydride in an ice bath, and then the pH value was adjusted to alkaline (pH 8) with 1 M sodium hydroxide, followed by reacting for 8 h in an ice bath. The reaction was dialyzed in water using 3500 dialysis bag at room temperature for 5 days, lyophilized, and then mixed with 5 mg of photoinitiator lithium phenyl-2,4,6-trimethylbenzoylphosphinate and 0.5 mL of water as the disperse phase, which was allowed to pass through microfluidic chip with the continuous phase (liquid paraffin containing 10% Span 80). The flow rate ratio of the disperse phase to the continuous phase was controlled to be 1:20. The hydrogel microspheres were collected, and then successively washed three times with 10% Tween-80 aqueous solution and PBS, to obtain 50 μm hydrogel microspheres (ECM);
2) 10 μL of ECM microspheres and 106 mesenchymal stem cells (MSCs) derived from bone marrow were allowed to co-assemble and cultured in cell plate for 24 h (temperature: 37° C., CO₂ concentration: 5%, medium: low-glucose DMEM medium containing 10% FBS and 1% penicillin-streptomycin), to obtain cell-microsphere co-assemblies (ECM-MSC).

### Example 2 Preparation of Cell-Microsphere Co-Assemblies According to the Present Invention

1) 0.05 g of gelatin was dissolved in 5 mL of water, to which was added 0.05 mL of methacrylic anhydride in an ice bath, and then the pH value was adjusted to alkaline (pH 9) with 1 M sodium hydroxide, followed by reacting for 12 h in an ice bath. The reaction was dialyzed at 30° C. for 5 days in water using 3500 dialysis bag, lyophilized, and then mixed with 3 mg of photoinitiator lithium phenyl-2,4,6-trimethylbenzoylphosphinate and 0.5 mL of water as the disperse phase, which was allowed to pass through microfluidic chip with the continuous phase (liquid paraffin containing 10% Span 80). The flow rate ratio of the disperse phase to the continuous phase was controlled to be 1:15. The hydrogel microspheres were collected, and then successively washed three times with 10% Tween-80 aqueous solution and PBS, to obtain hydrogel microspheres;
2) 5 μL of microspheres and 10⁷ tendon cells were allowed to co-assemble and cultured in cell plate for 18 h (temperature: 37° C., CO₂ concentration: 5%, medium: tendon cell medium containing 10% FBS and 1% penicillin-streptomycin), to obtain cell-microsphere co-assemblies.

### Example 3 Preparation of Cell-Microsphere Co-Assemblies According to the Present Invention

0.05 g of poly(ethylene glycol) diacrylate was mixed with 5 mg of photoinitiator lithium phenyl-2,4,6-trimethylbenzoylphosphinate and 0.5 mL of water as the disperse phase, which was allowed to pass through microfluidic chip with the continuous phase (liquid paraffin containing 10% Span 80). The flow rate ratio of the disperse phase to the continuous phase was controlled to be 1:25. The hydrogel microspheres were collected, and then successively washed three times with 10% Tween-80 aqueous solution and PBS, to obtain hydrogel microspheres;
2) 1 μL of microspheres and 10⁵ chondrocytes were allowed to co-assemble and cultured in cell plate for 24 h (temperature: 37° C., CO₂ concentration: 5%, medium: chondrocyte medium containing 10% FBS and 1% penicillin-streptomycin), to obtain cell-microsphere co-assemblies.

The beneficial effects of the present invention were further illustrated by reference to the following experimental example.

### Experimental Example 1 Study on the Treatment of Myocardial Infarction With the Decellularized Extracellular Matrix (Derived From Porcine Myocardial Tissue) and Mesenchymal Stem Cell Co-Assemblies

### I. Experimental Materials

### 1. Materials

Decellularized extracellular matrix derived from porcine myocardial tissue (d-ECM), methacrylic anhydride (MMA), lithium phenyl-2,4,6-trimethylbenzoylphosphinate (LAP), PBS, matrigel and the same.

Among them, d-ECM was prepared as follows: porcine myocardial tissue was collected and cut into small pieces with thickness of 1 mm, which were then stirred in PBS containing 1% SDS for 72 h, changing PBS every 24 h. After the tissue turns white, it was treated with 1% Triton X-100 solution for 30 min, and then washed with PBS for 3 days, followed by freeze-drying for future use. 10 mg of lyophilized decellularized myocardial tissue was digested in HCl solution (0.1 M) containing 1 mg of pepsin for 48 h, centrifuged to remove insoluble precipitates, adjusted the pH value to 7.4 with 1M NaOH, and then lyophilized to obtain the decellularized extracellular matrix (d-ECM) derived from porcine myocardial tissues.

### 2. Cells

Mesenchymal stem cells derived from the bone marrow of neonatal rats were cultured in DMEM medium (low glucose) containing 10% fetal bovine serum and 1% penicillin-streptomycin.

### 3. Animals

SPF-grade male SD rats, aged 6-8 weeks, were purchased from Beijing Vital River Laboratory Animal Technology Co., Ltd. All animal experiments were carried out under the approval and supervision of the Animal Management Committee of West China Hospital, Sichuan University.

### II. Experimental Methods

### 1. Preparation and Characterization of Microspheres and Cell-Microsphere Co-Assemblies

### 1.1 Preparation of Microspheres and Cell-Microsphere Co-Assemblies

Step 1: Preparation of microspheres. Using the decellularized extracellular matrix (d-ECM) derived from porcine myocardial tissue as the material, 0.05 g of d-ECM was dissolved in 5 mL of water, to which was added 0.05 mL of MAA in an ice bath, and then the pH value was adjusted to 8-9 with 1 M NaOH solution, followed by reacting overnight in an ice bath. Using 3500 dialysis bag, the reaction was dialyzed in water at room temperature for 5 days, and then lyophilized to obtain d-ECMMA for future use. Hydrogel microspheres were prepared from d-ECMMA and LAP by microfluidic technology (i.e., d-ECMMA was mixed with LAP and water as the disperse phase, and then passed through microfluidic chip, together with liquid paraffin containing 10% Span 80 as the continuous phase), and the flow rate ratio of the dispersed phase to the continuous phase was controlled to be 1:20. The hydrogel microspheres were collected, and then sequentially washed three times with an emulsifier aqueous solution (10% Tween-80 aqueous solution) and PBS, to obtain ECM hydrogel microspheres, which were characterized by microscope.
Step 2: Preparation of cell-microsphere co-assemblies. Using the prepared 50 μm microspheres (ECM) as the skeleton materials, 10 μL of ECM microspheres were allowed to co-assemble with 10⁶ mesenchymal stem cells (MSCs) derived from bone marrow and cultured for 24 h (temperature: 37° C., CO₂ concentration: 5%, medium: low-glucose DMEM medium containing 10% FBS and 1% penicillin-streptomycin), to obtain cell-microsphere co-assemblies (ECM-MSC).

### 1.2 Characterization of Microspheres and Cell-Microsphere Co-Assemblies

The prepared ECM-MSC was characterized by transmission electron microscopy (TEM) and microscopy, and the survival of MSCs in the co-assembly was investigated. In addition, the size of the cell-microsphere co-assembly was controlled by adjusting the amounts of cells and microsphere systems.

### 2. In Vitro and in Vivo Studies on Cell-Microsphere Co-Assemblies (ECM-MSC)

### 2.1 The Angiogenic Effect of ECM-MSC

The angiogenic effect of ECM-MSC was investigated using human umbilical vein endothelial cells (HUVEC). The specific procedure was as follows: Matrigel was placed in a 96-well plate and allowed to solidify. 3*10⁴ HUVEC cells were added, followed by addition of ECM-MSC secretion. The formation of tubules was observed at pre-determined time points.

### 2.2 Migration Effect of ECM-MSC

MSC cells were loaded into the ECM hydrogel microspheres prepared in Step 1 of the “1.1 Preparation of microspheres and cell-microsphere co-assemblies” section and used as the control, the migration ability of cells was investigated in Transwell chambers compared to that in the ECM-MSC prepared in Step 2 of the “1.1 Preparation of microspheres and cell-microsphere co-assemblies” section.

The preparation method for the control microspheres was as follows: The microspheres loaded with MSC cells were prepared from d-ECMMA, LAP, and MSC cells using microfluidic technology, with the same size as ECM-MSC.

### 2.3 in Vivo Animal Experiments

Using SPF-grade male SD rats as model animals, the treatment effect of ECM-MSC on myocardial infarction was studied. Firstly, a myocardial infarction model was established by ligating the left anterior descending coronary artery in rats. The treatment groups were as follows: (1) normal control group; (2) myocardial infarction group; (3) cell-microsphere assembly group, in which ECM-MSC was injected into the infarcted myocardial area through three intramyocardial injections. The experimental endpoints were to investigate the therapeutic effect of ECM-MSC on myocardial infarction using echocardiography, TCC staining, H&E staining, and Masson staining.

### III. Experimental Results

### 1. Characterization of Microspheres and Cell-Microsphere Co-Assemblies

### 1.1 Characterization of Microspheres

The microspheres prepared by microfluidic technology are shown in FIG. 1, with a size of 50 μm and uniform particle size distribution.

### 1.2 Characterization of Cell-Microsphere Co-Assemblies

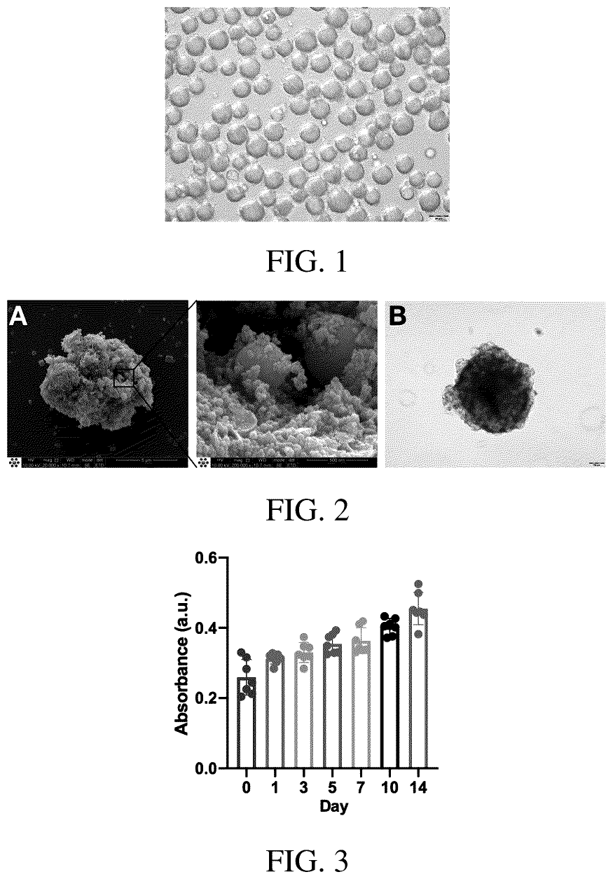

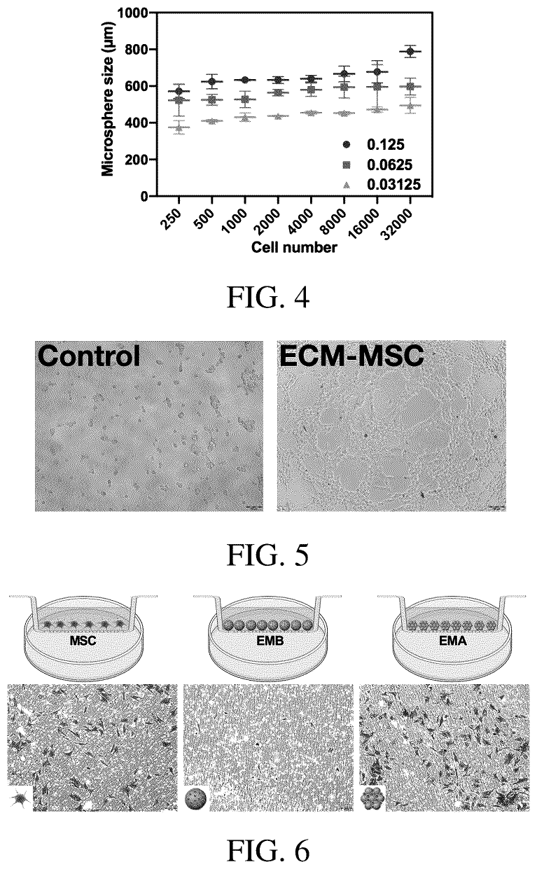

As shown in FIG. 2, microspheres were co-assembled with MSCs to obtain cell-microsphere co-assemblies with a size of 500 μm, wherein the cells tightly encapsulated the microspheres. The survival of MSCs in the ECM-MSC assembly was tested by CCK-8 assay, and the experimental results are shown in FIG. 3. The MSCs in the ECM-MSC assembly could proliferate with the extension of culture time, indicating that ECM in the ECM-MSC assembly could provide nutrients to promote the growth of MSCs. As shown in FIG. 4, microspheres of different sizes can be obtained by adjusting the amounts of cells and microsphere systems.

### 2. In Vitro and In Vivo Studies on Cell-Microsphere Co-Assemblies (ECM-MSC)

### 2.1 The Angiogenic Effect of ECM-MSC

The angiogenic effect of ECM-MSC was investigated by tube formation assay. The experimental results are shown in FIG. 5. HUVEC cells stimulated by ECM-MSC were able to form vascular network structures, indicating that ECM-MSC could release angiogenic-promoting factors by paracrine action, thereby promoting angiogenesis.

### 2.2 Migration Effect of ECM-MSC

The experimental results are shown in FIG. 6. Compared to large microspheres loaded with cells, the cell-microsphere assembly could better promote the migration of cells from the microspheres, which was more conducive to cell growth and proliferation.

### 3. In Vivo Animal Experiment

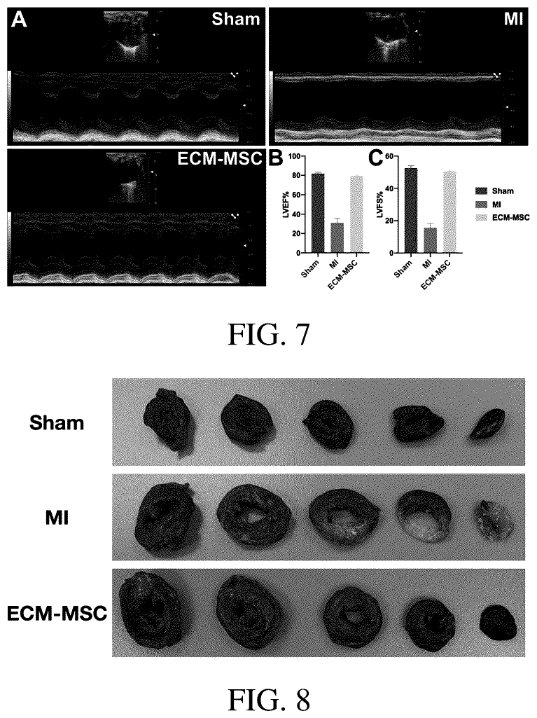

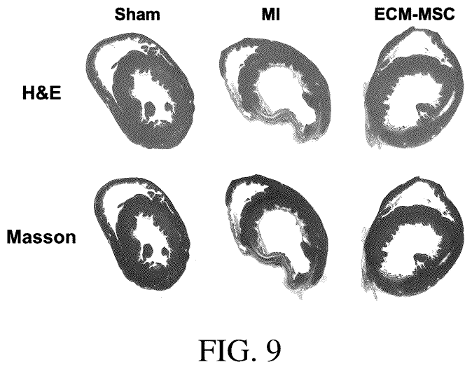

At the experimental endpoint, the therapeutic effect of ECM-MSC on myocardial infarction in rats was investigated using small animal ultrasound imaging. As shown in FIG. 7, after treatment with ECM-MSC, the myocardial infarction in rats could be effectively alleviated, with both ejection fraction and shortening fraction significantly higher than those in the MI group. TCC staining results (FIG. 8) revealed that the MI group had a large amount of infarcted tissues in the myocardium, while the ECM-MSC treatment group only had a small amount of infarcted tissues. The results of H&E staining and Masson staining (FIG. 9) further demonstrated that ECM-MSC could effectively alleviate infarction and inhibit the occurrence of fibrosis. This experimental result further indicated that the ECM-MSC system could improve cardiac function, reduce the infarct area, and decrease fibrosis in the infarct region, providing new ideas and practical approaches for the clinical treatment of myocardial infarction to promote public health, with positive social and economic benefits.

In summary, compared to the microsphere system where cells were encapsulated into hydrogel microspheres by blending cell and hydrogel microsphere materials, the cell-microsphere co-assembly ECM-MSC of the present invention was more conducive to cell proliferation and migration, and played sustained paracrine effects, showing more significant effects in promoting myocardial tissue repair.

1. A cell-microsphere co-assembly, characterized in that it is formed by the co-assembly of hydrogel microspheres and cells; the ratio of the hydrogel microspheres to cells is 0.0001-10 μL:10¹⁻⁷ cells;
the hydrogel microspheres are microspheres prepared by mixing any one or more of synthetic materials, natural materials, or derivative materials with photoinitiators;
the synthetic materials comprise polyethylene glycol, polyvinyl alcohol, povidone, polylactic acid, polycaprolactone, polyurethane, or polyglycerol sebacate;
the natural materials comprise gelatin, sodium alginate, sodium hyaluronate, chitosan, fibrin, collagen, or decellularized extracellular matrix (dECM);
the derivative materials comprise poly(ethylene glycol) diacrylate (PEGDA), methacryloylated poly(vinyl alcohol), methacryloylated polycaprolactone, methacryloylated polylactic acid, polyurethane methacrylate, gelatin methacryloyl (GelMA), sodium methacryloylated hyaluronate, sodium methacryloylated alginate, chitosan methacryloyl (CSMA), methacryloylated fibrin, methacryloylated collagen, or methacryloylated decellularized extracellular matrix.

2. The cell-microsphere co-assembly according to claim 1, characterized in that the hydrogel microspheres are microspheres prepared by mixing any one or more of synthetic materials, natural materials, or derivative materials with photoinitiators by means of microfluidic method, solvent evaporation method, phase separation method, spray drying method, electrostatic spraying method, templating method, or suspension polymerization method;
the photoinitiators comprise lithium phenyl-2,4,6-trimethylbenzoylphosphinate.

3. The cell-microsphere co-assembly according to claim 1, characterized in that the ratio of the hydrogel microspheres to cells is 10 μL:10⁶ cells.

4. The cell-microsphere co-assembly according to claim 1, characterized in that the cells comprise any one or more of mesenchymal stem cells, pluripotent stem cells, cardiomyocytes, vascular endothelial cells, adipose-derived stem cells (ADSCs), chondrocytes, and tendon cells, and mesenchymal stem cells are preferable.

5. The cell-microsphere co-assembly according to claim 1, characterized in that the particle size of the cell-microsphere co-assembly is 1-2000 μm; the particle size of the hydrogel microspheres is 1-1000 μm.

6. A method for preparing the cell-microsphere co-assembly according to claim 1, characterized in that it comprises the following steps:
1) Any one or more of synthetic materials, natural materials, or derivative materials are allowed to mix with a photoinitiator aqueous solution, and then passed through a microfluidic chip, to obtain hydrogel microspheres;
2) The hydrogel microspheres obtained in step 1) are mixed with cells according to the pre-determined ratio for co-assembly, to provide the cell-microsphere co-assembly.

7. The use of cell-microsphere co-assembly according to claim 1, in the manufacture of a medicament for tissue repair.

8. The use according to claim 7, characterized in that the tissue comprises cardiac muscle, skin, muscle, chondrocyte, blood vessels, tendons, and/or nerves.

9. The use according to claim 7, characterized in that the medicament is a local injection; the local injection has the effects of promoting neovascularization, inhibiting cell apoptosis, and accelerating tissue repair by locally injecting it into the damaged site, utilizing the paracrine function of cells.

10. The use according to claim 9, characterized in that the local injection comprises intramyocardial injection, pericardial cavity injection, coronary artery injection by catheter, intradermal injection, subcutaneous injection, intramuscular injection, joint cavity injection, vascular cavity injection, tendon sheath injection, intrathecal injection, and/or spinal nerve injection.
